(19) United States
(12) Patent Application Publication
(10) Pub. No.: US 2023/0310200 A1
(43) Pub. Date: Oct. 5, 2023

(54) DEVICE FOR ENHANCING AND/OR MAINTAINING A PENILE ERECTION AND/OR ENHANCING PLEASURE DURING SEXUAL ACTIVITY

(71) Applicant: 11304330 Canada Inc., Ottawa (CA)

(72) Inventor: Nigel A. SHAW, Ottawa (CA)

(21) Appl. No.: 18/012,046

(22) PCT Filed: Jun. 26, 2020
(86) PCT No.: PCT/CA2020/050888

§ 371 Date: Dec. 21, 2022

(51) Int. Cl. A61F 5/41

(52) U.S. Cl. CPC A61F 5/41; A61F 2005/414

(57) ABSTRACT

A device for treatment of erectile dysfunction is disclosed herein. The device has a stretchable ring to retract and maintain the foreskin of the penis a throughout sexual activity, and a stretchable tether coupled to the ring. An anchor end of the tether is mountable at a tailbone of the user, causing the tether to pull on the ring. The device is operative to increase the length of the sliding interface of the penis during sexual activity. The device operative to stimulate the stretch-sensitive nerve endings of the foreskin before and during sexual activity. The device increases stimulation of the sensitive nerve endings of the glans during sexual activity. The device retains blood within the penis upon erection, increasing the ability of the user to maintain an erection for a longer period and to achieve an erection more reliably. The device further increases the appearance of a larger penis.

### FIELD

The present disclosure pertains to the field of male sexual health. In particular, the present disclosure relates to a device for enhancing and/or maintaining a penile erection and optionally providing foreskin retraction or restraint.

### BACKGROUND

Erectile dysfunction (ED), also known as impotence, is a type of sexual dysfunction characterized by the inability to develop or maintain an erection of the penis during sexual activity. Erectile dysfunction can have psychological consequences as it can be tied to relationship difficulties and self-image. The most important organic causes of impotence are cardiovascular disease and diabetes, neurological problems (for example, trauma from prostatectomy surgery), hormonal insufficiencies (hypogonadism), and drug side effects.

First line treatment for ED include phosphodiesterase type 5-(PDE5) inhibitors such as sildenafil (Viagra), tadalafil (Cialis), and vardenafil (Levitra). Use of PDE5 inhibitors is contraindicated for men taking nitrate medications. In addition, a number of adverse drug reactions and drug interactions have been identified and as a result prevent the use of PDE5 inhibitors for the treatment of ED in some men. Moreover, the high cost of PDE5 inhibitors may further limit the availability of this treatment option for some affected men.

Currently available second-line treatment options include self-injection with vasoactive agents, vacuum erection devices, and surgical approaches with inflatable penile prostheses.

Other second-line treatment options include restricting blood flow out of the penis. The common penis ring or “cock ring” works in principle by this mechanism, however longitudinal movement of the ring along the shaft of the penis allows blood to escape, thereby reducing the effectiveness of the penis ring as a treatment for ED. To improve functionality of the penis ring, the penis ring may be made very tight. This however leads to discomfort and increased risk of injury. Variations of the penis ring are known in the art and include penis erection stabilizer disclosed in U.S. Pat. No. 6,319,194. Other erection aid devices include the simplified erection aid device disclosed in U.S. Pat. No. 5,728,043.

Both physical stimulation and erotic or emotional stimulation trigger and help maintain an erection. In non-circumcised or partially circumcised males, the foreskin slides, or “rolls” back and forth over the shaft of the erect penis during sexual stimulation to a greater or lesser extent depending on the amount of foreskin present. While the sliding and rolling of the foreskin has in and of itself a certain stimulating effect, it also has the potential to reduce overall penile stimulation. The foreskin alternately covers and uncovers the head, or glans, of the penis, and in some cases does not completely uncover the glans, thereby reducing the direct frictional stimulation of the glans, which is the most sensitive part of the penis. The foreskin further reduces the effective length of the sliding interface between the shaft of the penis and the inner wall of the vagina or alternate stimulating object or body part, on any insertion or retraction stroke by an amount equal to the length of the sliding of the foreskin. Devices for restraining penile foreskin are known in the art and include the device disclosed in U.S. patent application Ser. No. 10/325,598 (published as US 2011/0146695).

This background information is provided to reveal information believed by the applicant to be of possible relevance to the present invention. No admission is necessarily intended, nor should be construed, that any of the preceding information constitutes prior art against the present invention.

### BRIEF DESCRIPTION OF THE DRAWINGS

Embodiments will now be described, by way of example only, with reference to the attached Figures.

FIG. 1 shows a perspective view of a device for enhancing and/or maintaining a penile erection and/or enhancing pleasure during sexual activity according to embodiments of the disclosure, according to disclosed embodiments.

FIG. 2 shows a top plan view of the device of FIG. 1.

FIG. 3 shows an end elevation view of the device of FIG. 1.

FIG. 4 shows a side elevation view of the device of FIG. 1.

FIG. 5 shows a flowchart of a method of using the device of FIG. 1.

FIG. 6 shows an anterior view of a user wearing the device of FIG. 1, wherein hidden parts are shown in dashed lines.

FIG. 7 shows a posterior view of a user wearing the device of FIG. 1.

Throughout the drawings, sometimes only one or fewer than all of the instances of an element visible in the view are designated by a lead line and reference character, for the sake only of simplicity and to avoid clutter. It will be understood, however, that in such cases, in accordance with the corresponding description, that all other instances are likewise designated and encompassed by the corresponding description.

### DESCRIPTION

A device and system for enhancing and/or maintaining a penile erection and/or enhancing pleasure during sexual activity, and uses thereof, according to embodiments of the disclosure, are disclosed herein. The device and system are configured to be worn during sexual activity including intercourse or alternate penile stimulation and are designed to enhance the sexual experience.

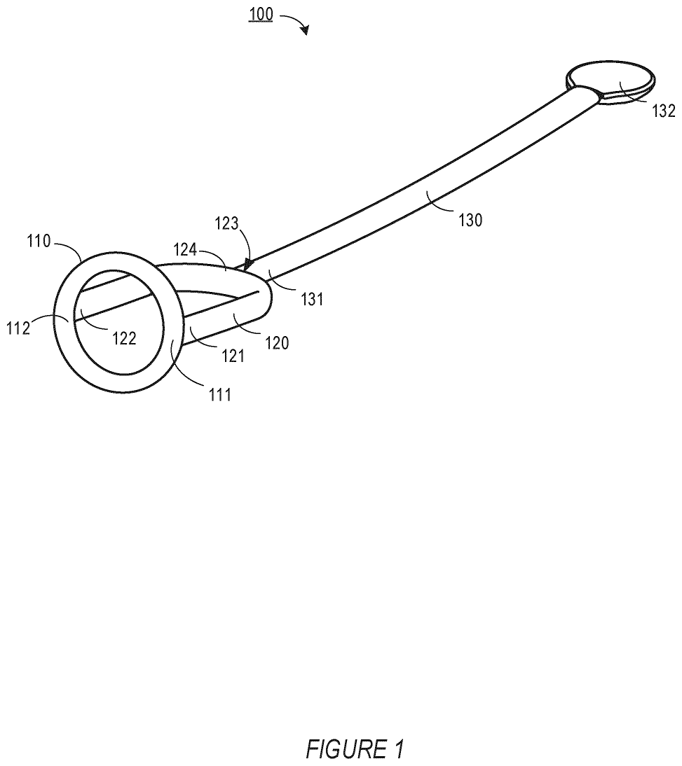

A device 100 according to embodiments of the invention is now described with reference to FIGS. 1-4.

The device 100 may have a stretchable substantially oval-shaped or substantially circular ring 110 sized such that, when placed around the base 211 of the penis 210 of a user 200, the ring 110 restricts blood flow out of the penis 210 without substantially impending blood flow into the penis 210. The device 100 may further have a stretchable loop 120 having ends 121, 122 attached to diametrically opposite sides of ring 110. When the loop 120 is placed around the scrotum 220 between the testicles 230 and the penis 210 as described and shown herein, it is neither loose nor tight. The ring 110 may be integral with the loop 120. The device 100 may further have a stretchable tether 130 attached to the loop 120 at a first end 131 of the tether 130. The tether 130 may be sized and shaped such that it reaches, when stretched, from the base of the scrotum 220 at the perineum (not shown), backward across the perineum, upward between the buttocks 240 at the base of the intergluteal cleft 250 and finally to the tailbone 260 or base of the spine. The loop 120 may be integral with the tether 130. A second, anchor end 132 of the tether 130 opposite the first end 131 may couple to an anchor 300 attached to the user 200. The anchor 300 may be any device operative to maintain the position of the anchor end 132 of the tether 130, and to maintain the tether 130 in a stretched condition. The anchor 300 may be or include a pad of approximately circular, oval, square or rectangular shape, approximately 1.5 to 2 inches in diameter, equipped with adhesive on one side thereof to adhere securely to human skin while allowing comfortable removal. The anchor end 132 of the tether 130 may be sized and shaped for optimal cooperation with the anchor 300, and in some embodiments may be substantially disc-shaped.

The device 100 and one or more anchors 300 may be provided as a kit, and may be used together as described, in which case they may form a system.

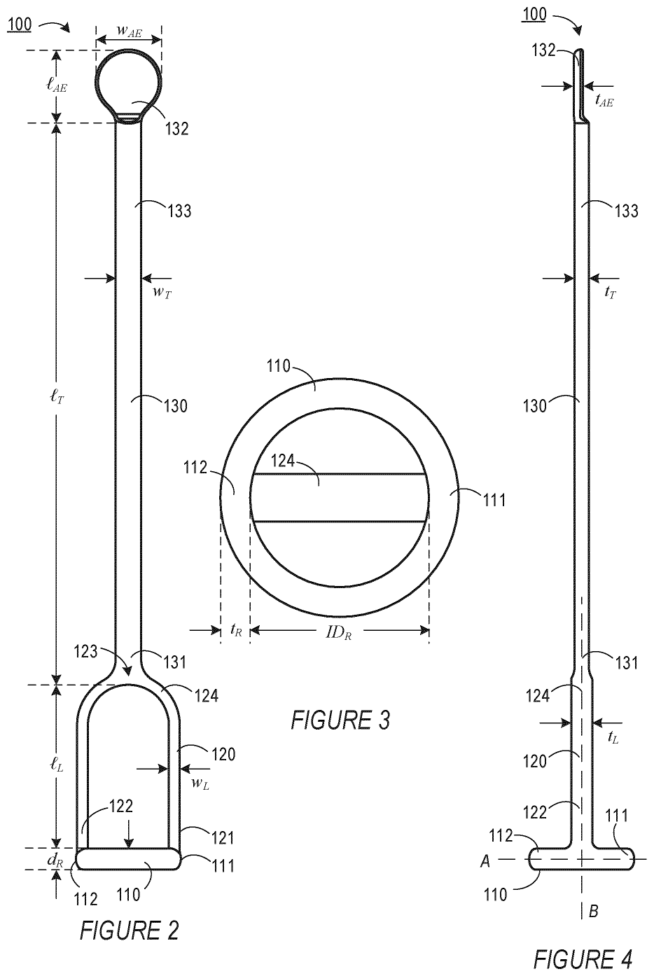

As shown particularly in FIG. 2, the loop 120 may be substantially U-shaped, and ends 121, 122 of the loop 120 may be attached at diametrically opposite sides 111, 112 of the ring 110, such that, as shown particularly in FIG. 3, a plane A of the ring 110 is substantially orthogonal to a plane B of the loop 120. As shown particularly in FIG. 2, the first end 131 of the tether 130 may be attached at the loop 120 at a midpoint 123 of a curved portion 124 of the loop 120. The anchor end 132 of the tether 130 and a face 310 of the anchor 300, which may be a non-adhesive side of the anchor 300, may be respectively equipped with cooperating parts of a two-part system of attachment and detachment, such as a snap system, a hook-and-loop system (e.g. Velcro™), or a hook system.

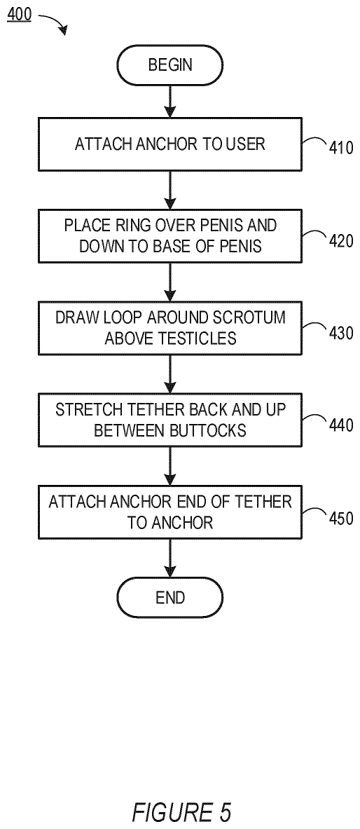

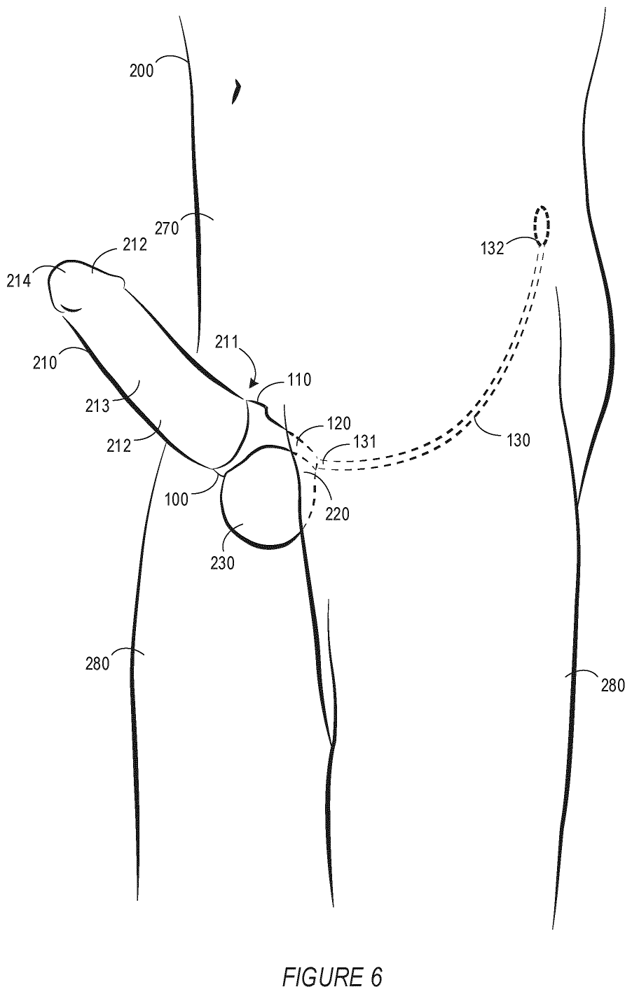

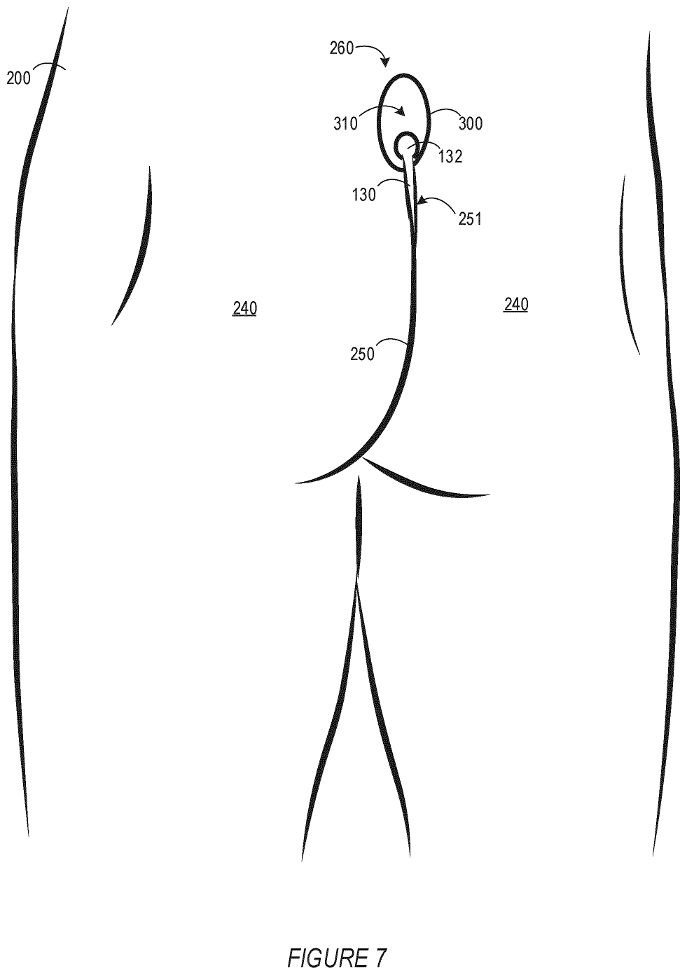

The device and system may be used for treating ED, and/or for enhancing and/or maintaining a penile erection and/or enhancing pleasure during sexual activity. For this purpose, the method 400 shown in FIG. 5 may be performed, as illustrated in FIGS. 6 & 7. As shown in FIG. 7, the anchor 300 (e.g. an adhesive pad) is attached to the user 200 at the base of the user's spine or on the tailbone 260 (step 410). With reference to FIG. 6, the device 100 is then worn by placing the ring 110 over the head 212 of the penis 210 and sliding the ring 110 down to the base 211 of the penis 210, drawing the loop 120 below the testicles 230 and then around the scrotum 220 above the testicles 230, and stretching the tether 130 back and up between the buttocks 240 and attaching the anchor end 132 of the tether 130 to the anchor 300.

More particularly, the device 100 may be held in front of the user 200 by the ring 110, with the plane of the ring 110 horizontal to a plane of the abdomen 270 of the user 200 and the attached loop 120 and the attached tether 130 extending downward. The ring 110 may be stretched and placed over the head 212 of the penis 210 and down to the base 211 of the penis 210 against the scrotum 220. The loop 120 may be stretched and drawn back and up over the testicles 230 to encircle the scrotum 220 above the testicles 230 and below the penile shaft 212. The tether 130 may be drawn rearward across the perineum (not shown), upward between the buttocks 240 such that it sits at the base 251 of the intergluteal cleft 250. The anchor end 132 of the tether 130 may attached to the anchor 300 such that the stretching tension of the tether 130—and therefore the pull on the ring 110 and loop 120—is maintained.

During use, the tether 130 may exert tension on the assembly of the ring 110 and loop 120, pulling the assembly rearward toward the base 211 of the penis 210. The scrotum 220 may thus be drawn rearward somewhat between the legs 280. The ring 110 may be drawn against the scrotum 220, preventing it from moving along the shaft 212 of the penis 210. The penile skin 213, by virtue of being attached to the rearwardly-drawn scrotum 220, may thus be stretched to be snug and to maintain the glans 214 uncovered during intercourse.

The device 100 may be made of any appropriate material including but not limited to elastic, latex, or silicone. In some embodiments, the device may be made of a single material and the parts are integral. Alternatively, the parts may be made as separate pieces and fused or otherwise attached. In other embodiments, the parts may be made from different materials.

The ring 110 may have an inner diameter ID_{R} of from about 22 mm to about 38 mm, a radial thickness t_{R} of from about 2 mm to about 6 mm, and a depth d_{R} of about 5 mm to about 15 mm. The loop 120 may have a thickness t_{L} of from about 2 mm to about 6 mm, a length l_{L} (as shown in FIG. 2, from the ends 121, 122 of the arms of the loop 120 to the inside surface of the midpoint 123 of the curved portion 124) from about 40 mm to about 70 mm (where a total length of the U-shape of the loop 120, i.e. from one end 121 through the curved portion 124 to the other end 122 may be from about 100 mm to about 180 mm), and arms of the loop 120 may each have a width w_{L} of from about 5 mm to about 15 mm. A body 133 of the tether 130 may have a thickness t_{T} of from about 2 mm to about 6 mm (which may be the same or different from the thickness t_{L} of the loop 120), a length l_{T} of from about 120 mm to about 200 mm, and a width w_{T} of from about 5 mm to about 10 mm. The anchor end 132 of the tether 130 may have a thickness t_{AE} of from about 2 mm to about 5 mm (which may be the same or different from the thickness t_{T} of the body 133 of the tether 130), a length l_{AE} of from about 10 mm to about 30 mm, and a width w_{AE} of from about 10 mm to about 30 mm. Other dimensions are possible.

The device 100, by slowing blood flow out of the penis 210 from the base 211 of the penis 210 and drawing the penile skin 213 rearward to stimulate the stretch-sensitive nerve endings, facilitates the start and maintenance of an erection. The ring 110 of the device 100 exerts a compressive force at the base 211 of the penis 210 thereby partially restricting blood flow from the penis 210 resulting in a more reliable, harder and more lasting erection. The device 110 may apply pressure to the dorsal veins (not shown), both the superficial dorsal and the deep dorsal and may also provide supportive pressure on the two corpus cavernosa helping to enable adequate compressive force.

The device 100, by holding the ring 110 in place at the base 211 of the penis 210, allows for the ring 110 to be of a more comfortable and safe size while still being effective for treating erectile dysfunction, and/or for enhancing and/or maintaining a penile erection and/or enhancing pleasure during sexual activity.

The device and system provide a number of distinct benefits over those provided by a normal constriction ring (penile ring or “cock ring”), as follows.

Normal constriction rings tend to move laterally along the shaft of the penis during sexual activity. Lateral movement along the shaft causes a normal constriction ring to squeeze blood through the vessels it is constricting, expelling trapped blood from the penis and greatly diminishing its efficacy at trapping blood. Because the ring of the device disclosed herein is securely held at the base of the penis, the ring is prevented from moving laterally along the shaft of the penis. The device therefore traps blood more efficiently in the penis, enhancing the erection.

By virtue of the device's enhanced ability to trap blood, the device promotes engorgement of the penile shaft subcutaneous tissues, making the penis slightly thicker, and making the penis look and feel thicker and harder.

The device may pull the penile skin backward, which stimulates the special stretch-sensitive nerve endings of the penile skin, triggering the erection response and helping to maintain the erection.

The device's unique design optimizes frictional sensation. The effective frictional length of the intercourse stroke is increased because the stroke no longer has to take up the slack in the foreskin. This enhances sensation for both partners.

By maintaining the penile skin in a retracted position, the device optimizes glans exposure, increasing stimulation of the most sensitive area of the penis.

The device draws the testicles and scrotum rearward, optimizing shaft penetration length and making the penis appear longer.

The device, by encircling the scrotum above the testicles with the loop, prevents uncomfortable testicle “ride-up,” in which the testicles move up on either side of the penis and are squeezed between the man and his partner.

The device and system may also be used for the treatment of urinary incontinence, such as climacturia.

The following are examples according to the disclosure herein.

Example 1. A device for use with human male genitalia, the device comprising: a stretchable ring; and a stretchable tether coupled to the ring, the tether having a first end proximal the ring, an anchor end opposite the first end and distal the ring, and a body spanning the first end and the anchor end.

Example 2. The device of Example 1, wherein the ring is sized and shaped to be placed fittingly around a base of a penis of the genitalia to restrict blood flow out of the penis without substantially impeding blood flow into the penis.

Example 3. The device of Example 1, wherein the ring constricts the base of the penis.

Example 4. The device of any one of Examples 1 to 3, wherein the anchor end is sized, shaped, and configured for coupling with an anchor wearable by a user.

Example 5. The device of any one of Examples 1 to 4 further comprising: a stretchable loop attached to the ring; wherein the tether is attached to the loop at the first end of the tether.

Example 6. The device of Example 5, wherein the loop is sized and shape to encompass a root of a scrotum of the genitalia.

Example 7. The device of Example 6, wherein the loop is sized and shaped for placement of a curved portion of the loop behind the scrotum.

Example 8. The device of any one of Examples 5 to 7, wherein the loop is U-shaped, and opposing ends of the loop are attached at corresponding diametrically opposite sides of the ring.

Example 9. The device of Example 8, wherein a plane of the loop is substantially orthogonal to a plane of the ring.

Example 10. The device any one of Examples 1 to 9, wherein the anchor end of the tether has a thickness different from a thickness of the body of the tether.

Example 11. The device of any one of Examples 1 to 10, wherein the anchor end of the tether has a width different from a width of the body of the tether.

Example 12. The device of any one of Examples 1 to 9, wherein the tether has a width and a thickness so as to fit within a human intergluteal cleft.

Example 13. The device of any one of Examples 1 to 12, further comprising an attachment device mounted at the anchor end of the tether.

Example 14. The device of Example 13 or 14, wherein the attachment device comprises at least a part of a snap system, a hook-and-loop system, or a hook system.

Example 15. The device of Example 13, where the attachment device is configured to attach to an anchor mountable on a human user.

Example 16. A system comprising the device of Example 15 and the anchor defined by Example 15.

Example 17. The system of Example 16, wherein the anchor is mountable at a base of a spine or at a tailbone of a user.

Example 18. A kit comprising the device of Example 15, the anchor defined by Example 15, and instructions.

Example 19. A method of using the system of Example 16 or 17, the method comprising: attaching the anchor to the user at a base of the spine or the tailbone of the user; placing the ring over a head of the penis and sliding the ring down to a base of the penis; stretching the tether between buttocks of the user and attaching the anchor end of the tether to the anchor.

Example 20. The method of Example 19, wherein when the device is mounted the tether is in tension and pulls on the ring.

Example 21. The method of Example 19 or 20 when dependent on Example 5, further comprising, after sliding the ring down to a base of the penis and before stretching the tether between buttocks: drawing the loop below testicles of the genitalia and around the scrotum above the testicles so as to encompass the root of the scrotum.

Example 22. Use of the device of any one of Examples 1 to 15, the system of Example 16 or 17, the kit of Example 18, or the method of any one of Examples 19-21, to treat erectile dysfunction or climacturia.

Unless defined otherwise, all technical and scientific terms used herein have the same meaning as commonly understood by one of ordinary skill in the art to which embodiments of the invention pertain. Methods and materials similar, modified, or equivalent to those described herein can be used in the practice of the embodiments of the present invention without undue experimentation.

Numeric ranges recited within the specification are inclusive of the numbers defining the range and include each integer within the defined range. Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numbers within that range, for example, 1, 2, 3, 4, 5, and 6, and decimals and fractions, for example, 1.2, 3.8, 1½, and 4¾. This applies regardless of the breadth of the range.

The term “about,” as used herein, refers to variation in the numerical quantity that can occur, for example, through typical measuring techniques and equipment, with respect to any quantifiable variable. Further, given handling procedures used in the real world, there is certain inadvertent error and variation that is likely through differences in the manufacture, source, or purity of the materials used. The term “about” also encompasses these variations.

In the preceding description, for purposes of explanation, numerous details are set forth in order to provide a thorough understanding of the embodiments. However, it will be apparent to one skilled in the art that these specific details are not required. In particular, it will be appreciated that the various additional features shown in the drawings are generally optional unless specifically identified herein as required. The above-described embodiments are intended to be examples only. Alterations, modifications and variations can be effected to the particular embodiments by those of skill in the art. The scope of the claims should not be limited by the particular embodiments set forth herein, but should be construed in a manner consistent with the specification as a whole.

1. A device for use with human male genitalia, the device comprising:
a stretchable ring; and
a stretchable tether coupled to the ring, the tether having a first end proximal the ring, an anchor end opposite the first end and distal the ring, and a body spanning the first end and the anchor end.

2. The device of claim 1, wherein the ring is sized and shaped to be placed fittingly around a base of a penis of the genitalia to restrict blood flow out of the penis without substantially impeding blood flow into the penis.

3. The device of claim 1, wherein the ring constricts the base of the penis.

4. The device of claim 1, wherein the anchor end is sized, shaped, and configured for coupling with an anchor wearable by a user.

5. The device of claim 1 further comprising:
a stretchable loop attached to the ring;
wherein the tether is attached to the loop at the first end of the tether.

6. The device of claim 5, wherein the loop is sized and shaped to encompass a root of a scrotum of the genitalia.

7. The device of claim 6, wherein the loop is sized and shaped for placement of a curved portion of the loop behind the scrotum.

8. The device of claim 5, wherein the loop is U-shaped, and opposing ends of the loop are attached at corresponding diametrically opposite sides of the ring.

9. The device of claim 8, wherein a plane of the loop is substantially orthogonal to a plane of the ring.

10. The device of claim 1, wherein the anchor end of the tether has a thickness different from a thickness of the body of the tether.

11. The device of claim 1, wherein the anchor end of the tether has a width different from a width of the body of the tether.

12. The device of claim 1, wherein the tether has a width and a thickness so as to fit within a human intergluteal cleft.

13. The device of claim 1, further comprising an attachment device mounted at the anchor end of the tether.

14. The device of claim 13, wherein the attachment device comprises at least a part of a snap system, a hook-and-loop system, or a hook system.

15. The device of claim 13, where the attachment device is configured to attach to an anchor mountable on a human user.

16. A system comprising the device of claim 15 and the anchor defined by claim 15.

17. The system of claim 16, wherein the anchor is mountable at a base of a spine or at a tailbone of the user.

18. (canceled)

19. A method of using the system of claim 16, the method comprising:
attaching the anchor to the user at a base of the spine or the tailbone of the user;
placing the ring over a head of the penis and sliding the ring down to a base of the penis;
stretching the tether between buttocks of the user and attaching the anchor end of the tether to the anchor.

20. The method of claim 19, wherein when the device is mounted the tether is in tension and pulls on the ring.

21. The method of claim 19, wherein the device further comprises:
a stretchable loop attached to the ring;
wherein the tether is attached to the loop at the first end of the tether;
the method further comprising, after sliding the ring down to a base of the penis and before stretching the tether between the buttocks:
drawing the loop below testicles of the genitalia and around the scrotum above the testicles so as to encompass the root of the scrotum.

22. (canceled)
